(19) United States
(12) Patent Application Publication
(10) Pub. No.: US 2018/0133368 A1
(43) Pub. Date: May 17, 2018

(54) 3D Printed Ti-6Al-4V Scaffolds with Hydrogel Matrix

(71) Applicants: Raja Devesh Kumar Misra, El Paso, TX (US); Alok Kumar, El Paso, TX (US); Krishna Chaitanya Nune, El Paso, TX (US); Lawrence E. Murr, El Paso, TX (US)

(72) Inventors: Raja Devesh Kumar Misra, El Paso, TX (US); Alok Kumar, El Paso, TX (US); Krishna Chaitanya Nune, El Paso, TX (US); Lawrence E. Murr, El Paso, TX (US)

(73) Assignee: THE BOARD OF REGENTS OF THE UNIVERSITY OF TEXAS SYSTEM, Austin, TX (US)

(21) Appl. No.: 15/672,680

(22) Filed: Aug. 9, 2017

Related U.S. Application Data
(60) Provisional application No. 62/422,158, filed on Nov. 15, 2016.

(51) Int. Cl. A61L 27/52; A61L 27/56; A61L 27/54; A61L 27/20; A61L 27/12; A61L 27/36; A61L 27/38

(52) U.S. Cl. CPC A61L 27/52; A61L 27/56; A61L 27/54; A61L 27/20; A61L 27/12; A61L 2430/02; A61L 27/3821; A61L 2300/414; A61L 2300/406; A61L 2300/204; A61L 2400/06; A61L 27/3633

(57) ABSTRACT

Embodiments of the invention are directed to a vascular structure forming implant produced by additive manufactured Ti-6Al-4V scaffolds a living implant.

### CROSS REFERENCE TO RELATED APPLICATIONS

This application claims priority to U.S. Provisional Application 62/422,158 filed Nov. 15, 2016, which is incorporated herein by reference in its entirety.

### BACKGROUND OF THE INVENTION

### I. Field of the Invention

The invention generally concerns an injectable hydrogel and methods of using the same. In particular the injectable hydrogel includes osteoblast or osteoblast precursor cells.

### II. Description of Related Art

3D printing or Additive Manufacturing (AM) has revolutionized the way materials scientists and engineers synthesize a broad spectrum of materials. This technology allows bioengineers to enhance metals, composites, polymer plastics, and biomedical devices. Tissue engineering is an emerging field in which materials that display biomimetic properties are employed for medical applications. Despite the considerable amount of research advances being made (Surmenev et al., Acta Biomaterialia, 2014, 10:557-79; Bosch et al., Journal of Craniofacial Surgery, 1998, 9:310-6; Meinel et al., Bone, 2005, 37:688-98; Gugala and Gogolewski, Injury, 2002, 33:71-6; Schütz and Südkamp, Journal of orthopaedic science, 2003, 8:252-8; Kumar et al. Materials Science & Engineering R: Reports, 2016, 103:1-39), the main challenge in tissue engineering is to create a fully functional living system parting from a non-living scaffold. There exists an ever present need to develop materials that are not just bio-compatible, but that can more closely mimic a complete biological system.

Living tissue is comprised of complex interactions between different cell types, all of which perform different tasks, depending on the cell type and the type of tissue they happen to be present in. Due to the highly regulated cell biology processes, complexity of molecular interactions and cellular differentiation hierarchy, engineering tissue remains a challenging endeavor. Bone is composed mainly of mineralized calcium crystals (hydroxyapatite), with a chemical formula of Ca₅(PO4)₃(OH), and collagen. The mixture of these structures provides mechanical support and a degree of elasticity (Kumar et al., International Materials Reviews, 2016, 61:20-45). Despite the success of implanted orthopedics, mainly hip-replacement implants, there has been evidence of infection, aseptic loosening, pain without loosening or other reasons of failure (Diefenbeck et al., Biomaterials, 2011, 32:8041-7). When a bone replacement implant is inserted, a surgeon needs to cut an extensive amount of bone, effectively creating a wound. Cells at the interface between the bone and the implant cannot grow into the solid bio-compatible metal alloy. Bone replacement implants remain as solid structures, providing a physical limitation for cells to grow.

### SUMMARY OF THE INVENTION

Many strategies have been employed in order to enhance the bio-compatibility of orthopedic implants (Heimann et al., J Mater Sci: Mater Med., 2004, 15:1045-52; Frenkel et al., Journal of Biomedical Materials Research, 2002, 63:706-13; Tagai and Aoki, Preparation of synthetic hydroxyapatite and sintering of apatite ceramics: John Wiley and Sons; 1980; Kumar et al., Journal of Biomedical Materials Research Part B: Applied Biomaterials, 2013, 101B:223-36; Kumar et al., Acta Materialia, 2013, 61:5198-215; Kumar et al., Journal of biomaterials applications, 2016, 0885328216658376), but a more diverse set of materials needs to be examined in order to closely mimic a complete living system. To this end, 3D printed scaffolds are an effective alternative, given their degree of porosity, in particular Ti-6Al-4V printed scaffolds. Advantages to this approach include varying pore size gradient, varying porosity, and a high degree of resolution control on the implant synthesis. An extracellular matrix-like gel in combination with 3D printed scaffolds was evaluated for the development of a bone replacement implant. In this research, a Ti-6Al-4V scaffold structure was designed to promote cell migration of vascular endothelial cells, and differentiation and proliferation of pre-osteoblast cells. Given that there is a degree of porosity in these scaffolds, a matrix can be applied to the structure; allowing for microcapillary formation in a 3D suspension. Since hydrogels are highly hydrated polymers, certain molecules and growth factors can be mixed into it, providing cells with the necessary supplements required for proliferation and growth.

Certain embodiments are directed to a living implant. A living implant is one that creates a living replacement of bone tissue. In certain aspects a structural Ti-6Al-4V scaffold can be used in combination with a hydrogel material containing a stress inducer. Portions of the hydrogel will be catalyzed, metabolized, or degraded by surrounding tissue and eventually replaced by said tissue. An argument can be made that the ingrowth of bone presents an answer to stress shielding effects of modern day implants. A stress inducer can enhance the production of hydroxyapatite and bone density should not decrease as a result.

Hydrogels have been widely employed in cell culture environments, with varied applications (Kumar et al., Journal of biomaterials applications, 2016, 0885328216658376; Kumar et al. Materials Science and Engineering C, 2012, 32:464-9). Many materials have been examined and tested for applications in the biomedical field, but one of the most promising materials have been gelatin based hydrogels (Kumar et al. Materials Science and Engineering C, 2012, 32:464-9; Yuksel et al., International Journal of Pharmaceutics, 2000, 209:57-67; Kumar et al., Journal of Biomedical Materials Research Part A, 2013, 101:2925-38). Developing a 3D matrix to grow cells in is advantageous in many ways, given that it has been previously shown (Kumar et al., Journal of biomaterials applications, 2016, 30:1505-16) that a 2D environment does not fully mimic physiological conditions. This is because the 3D matrix will allow cells to behave as they would in a normal physiological environment morphologically and also enhancing cell-cell communication (Rowley et al., Biomaterials, 1999, 20:45-53).

Certain embodiments are directed to a hydrogel comprising one or more of alginate, gelatin, nanocrystalline hydroxyapatite, 1-ethyl-3-(3-dimethylaminopropyl) carbodiimide (EDC), N-hydroxysuccinimide (NHS), and/or osteoblast or osteoblast precursor cells, or the various combinations thereof. In certain aspects the hydrogel can contain 0.005, 0.01 to 0.015, 0.02 g/ml alginate, in certain instance 0.01 g/ml alginate. In a further aspect the hydrogel can contain 0.005. 0.01 to 0.015, 0.02 g/ml gelatin, in certain instance 0.01 g/ml gelatin. In still a further aspect the hydrogel contains 1, 2, 3, 4, 5 to 6, 7, 8, 9, 10 mg/ml nanocrystalline hydroxyapatite, in certain instances about 5 mg/ml nanocrystalline hydroxyapatite. The hydrogel will comprise water to volume. In certain aspects the hydrogel contain osteoblast or osteoblast precursor cells. The hydrogel can be crosslinked using CaCl₂. In certain aspects the hydrogel can contain 2 to 3 mg/ml EDC and 1 to 2 mg/ml NHS. In certain aspects nanocrystalline hydroxyapatite is in the form of particles. The hydroxyapatite particles can be needle shaped or elongated having a length or average length of about 50, 60, 70, 80, 90, 100 nm or so and a diameter of about 10, 20, 30, 40, 50 nm or so.

Alginate is an anionic polysaccharide distributed widely in the cell walls of brown algae, where through binding with water it forms a viscous gum. Alginate is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks) or alternating M and G-residues (MG-blocks). In certain aspects other anionic polysaccharide can be used in place of alginate.

Gelatin is a mixture of peptides and proteins produced by partial hydrolysis of collagen extracted from the skin, bones, and connective tissues of animals such as domesticated cattle, chicken, pigs and fish. During hydrolysis, the natural molecular bonds between individual collagen strands are broken down into a form that rearranges more easily. Its chemical composition is, in many respects, closely similar to that of its parent collagen.

Other embodiments are directed to an implant or a bone replacement implant comprising (a) a three dimensional support; and (b) a hydrogel matrix comprising a hypoxia inducer and glucose, wherein the implant is capable of promoting vascularization and osteogenesis. In certain aspects the three dimensional support is a scaffold structure of Ti-6Al-4V or similar alloy. The scaffold structure can have a porosity of 50 to 70%, in particular 60%. The scaffold structure can have an average pore size of 200, 300 to 400, 500 μm, in particular about 350 μm. In certain aspects the scaffold structure has a thickness of 0.25 to 5 cm. The density of the scaffold structure can be between 0.5 to 3 g/cm² or 1 to 2 g/cm² or in particular about 1.5 g/cm². In certain aspects the hypoxia inducer is deferoxamine mesylate (DFM). DFM can be present in the hydrogel at a concentration of about 0.5, 1, 2 to 5, 10, 15 μM, including all values and ranges there between. In certain aspects DFM concentration can be as high as 2 to 10 mM. The hydrogel can comprise natural, synthetic, or natural and synthetic polymers. In certain aspects the hydrogel can comprises proteins of the extracellular matrix, particularly collagen. Natural polymers can include one or more of polyhyaluronic acid, alginate, polypeptides, collagen, elastin, polylactic acid, polyglycolic acid, or chitin. Synthetic polymers can include one or more of methacrylated gelatin, polyethylene oxide, polyethylene glycol, polyvinyl alcohol, polyacrylic acid, polyacrylamide, poly(N-vinyl-2-pyrrolidone), polyurethane, or polyacrylonitrile. In certain aspects the hydrogel further comprises one or more growth factors or antibiotic.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B. (A) Schematic of hydrogel preparation and (B) infiltration of cell-loaded hydrogel in 3D printed Ti-6Al-4V porous scaffold.

FIGS. 2A-2F. (A-C) Representative scanning electron micrographs of freeze-dried hydrogel at different magnifications, (D) histogram showing the pore-size distribution, and (E-F) results of EDS analysis.

FIG. 3. XRD of freeze-dried hydrogel, its components (hydroxyapatite, alginate, and gelatin), and base material (perspex) used to hold the sample during XRD.

FIG. 4. FT-IR of HA, alginate, gelatin, and freeze-dried hydrogel. KBr was used as a reference material.

FIG. 5. Representative data showing the variation in the hydrogel swelling ratio with time in 1× PBS at 37° C. at constant humidity maintained in hot air oven.

FIG. 6. Representative data showing the variation in the hydrogel swelling rate with time in 1× PBS at 37° C. at constant humidity maintained in hot air oven.

FIG. 7. Representative data showing the variation in desorption rate of 1× PBS from hydrogel with time at 37° C. at constant humidity maintained in hot air oven.

FIGS. 8A-8C. (A) Mechanism of cross-linking of alginate and gelatin through EDC and EHS. (B) Mechanism of cross-linking of alginate and gelatin through CaCl₂. (C) Schematic of cross-linking of alginate and gelatin with EDC, NHS, and 0.05M CaCl₂.

FIG. 9. Standard plot between dissolved alginate in 1× PBS and absorption at 210 nm to obtain relationship between absorption and dissolved amount of alginate.

FIGS. 10A-10B. (A) Effect of immersion time on the dissolution behavior of hydrogel and (B) representative photograph of hydrogel after 8 days of immersion in 1× PBS.

FIGS. 11A-11C. (A) Representative low magnification (i,ii) and high magnification (iii,iv) fluorescence micrographs, showing the effect of 5 min cross-linking on the cell viability (i,iii) and necrotic cell death (ii,iv). (B) Representative low magnification (i,ii) and high magnification (iii,iv) fluorescence micrographs, showing the effect of 10 min cross-linking on the cell viability (i,iii) and necrotic cell death (ii,iv). (C) Representative low magnification (a,b) and high magnification (c,d) fluorescence micrographs, showing the effect of 15 min cross-linking on the cell viability (a,c) and necrotic cell death (b,d).

FIG. 12. Effect of cross-linking time on the thickness of the cross-linked layer and its stability in culture medium.

FIGS. 13A-13H. (A-C) Representative low magnification fluorescence micrographs showing the staining for nucleus, vinculin, and actin filament. (D-F) High magnification micrographs of images a, b, and c. (G) Three dimensional morphology of osteoblasts in hydrogel matrix. (H) Elongated morphology of osteoblasts growing in contact with Ti-6Al-4V scaffold strut.

FIG. 14. Results of MTT assay of cells grown for 2, 4, and 8 days on hydrogel and well plate surface. Data are reported as mean±standard error of mean (n=3). The * showing the significant difference in optical density when control was compared with hydrogel using Dunnett t test at p<0.05. The ** showing the significant differenece in optical density at p<0.05 when comparison was made among the samples (Dunnett C test).

FIGS. 15A-15B. (A) Hydrogel after MTT assay, showing the absorption of formazan crystals in hydrogel. (B) Schematic of cells grown on the hydrogel surface and absorption of culture medium on the hydrogel.

FIG. 16. Results of ALP assay of osteoblasts grown on control and hydrogel surface for 4 days, followed by differentiation for 6 and 12 days. Data is reported as mean±standard error of mean (n=3). The * showing the significant difference in optical density when control was compared with hydrogel using Dunnett t test at p<0.05. The ** showing the significant differenece in optical density at p<0.05 when comparison was made among the samples (Dunnett C test).

### DETAILED DESCRIPTION OF THE INVENTION

Scaffolds provide an ideal substrate for substitute bone due to their random distribution and interconnection, which is largely similar to that of real bone. Ti-6Al-4V has been a popular alloy used in the biomedical industry and research and has been extensively characterized. The limitation of free iron availability through exposure of DFM seems to be a driving factor to enhance the synthesis of hydroxyapatite by cells. It has been previously demonstrated that pre-osteoblasts proliferate, differentiate and are able to synthesize hydroxyapatite when grown on scaffold and mesh structures of this alloy. However, described herein for the first time is an implant that supports formation of a vascular network in the context of a scaffold alloy.

The process of vascular structure initiation has a key step that involves proteolytic degradation of the ECM so that endothelial cells can migrate to form the microcapillarities. DFM has proven to be a suitable candidate molecule to promote vascularization of endothelial cells. Immediate biomedical applications of this iron chelating agent are viable, seeing as it is already been FDA approved for the treatment of iron poisoning. Described herein is the concept of a living implant as it pertains to various cellular molecular mechanisms, mainly involved in wound healing and the regeneration of tissue. Tissue that has undergone extensive damage needs to endure harsh environments that stimulate apoptosis rather than cell survival. A tissue-solid metal interface is not sufficient to promote a wound healing process, the ingrowth of bone, and eventually the formation of a vascular network. Implanted solid metal bars present a physical limitation to the availability of nutrients and, most notably, oxygen. The wounded tissue then suffers from hypoxia, triggering an irreversible response that eventually leads to cellular death. In a heavily wounded tissue metabolic demands differ vastly from that of normal tissue. To create a fully living implant that mimics real tissue, this issue needs to be addressed and thoroughly understood. Therefore, the addition of molecules that can compensate for the metabolic high demands is required. It is to this end that D(+) glucose can be added to cells undergoing a hypoxia mimetic response.

The materials chosen and tested herein prove to be a combination that is suitable to develop a fully living bone replacement implant. Ti-6Al-4V scaffolds provide structural support, while an ECM-like hydrogel simulates an aqueous microenvironment that drives wound healing, bone ingrowth and vascularization. Despite the attractive properties of Matrigel, this product is not intended for anything other than research purposes. However, its main constituents may be further utilized with the focus of creating a hydrogel capable of driving the before mentioned processes. Collagen and gelatin hydrogels can be tailored to maintain their solid stability under physiological conditions.

Embodiments of the invention include materials comprising a base support coupled to a hydrogel that includes reagents for supporting vascularization and enhancing bone repair, while maintaining mechanical and structural similarities with real bone. Certain aspects are directed to a mixture of additive manufactured Ti-6Al-4V scaffold in combination with a collagen based hydrogel matrix containing DFM; a hypoxia mimetic compound, that can form vasculature under physiological conditions, while maintaining osteoblast cell differentiation and proliferation. This approach induces a hypoxia mimetic stress that will trigger cellular survival signals that ultimately enhances wound healing processes in bone.

### I. STRUCTURED SCAFFOLD SUPPORT

A structured scaffold can be rapidly built from a base powder material. For example, the scaffold structure can be manufactured using three-dimensional (3D) printing. A direct metal laser sintering process can be used to 3D print (i.e. build) the scaffold structure. The scaffold structure can be made from a base material, such as a Ti-6Al-4V alloy. In other embodiments the scaffold structure can comprise other suitable alloys or combination of alloys (e.g., 316 stainless steel, commercially pure titanium (TiCP) and aluminum alloy (AlSi10Mg), austenitic steels, ferrous steels, aluminum alloys, titanium alloys, pure aluminum and pure titanium and the like).

The scaffold structure can be three-dimensionally printed with a direct metal laser sintering process (or any other suitable process, such as electron beam melting). The scaffold structure can be three-dimensionally printed with at least a Ti-6Al-4V alloy (or any other suitable alloy or combination of alloys). The scaffold structures can be produced using electron beam melting or any other additive manufacturing process.

### II. EXTRACELLULAR MATRIX-LIKE HYDROGELS FOR BONE REPAIR

The ingrowth of bone into the implant is essential in order to achieve what is conceptually a living implant. Although the main goal of this research is to stimulate the formation of vascular structures within the porous metal implant, the nature of wound healing must also be addressed. This includes, but is not limited to the mineralization of calcium by osteoblasts, the inhibition of bone resorption by osteoclasts, and avoiding debris release by the implant itself. Bone has elastic properties, and its elasticity can be attributed to the collagen in which hydroxyapatite grows. The molecular arrangement of collagen is regulated by fibroblasts and endothelial cells in tissue, and this arrangement directs the synthesis and growth of hydroxyapatite. Bone is capable of self-repair, but this natural process is limited to the extents to which it can generate new tissue. This is the case for the large portion of bone that has been surgically removed. However, with the assistance of engineered biomaterials, bone tissue repair can be directed by stimulating the appropriate wound healing response. The microenvironment of bone has been widely reported to be hypoxic. A hypoxia mimetic environment has been reported to enhance bone repair, along with restoring endothelium integrity. This was determined by injecting DFM on mandibular fractures that had been exposed to radiotherapy. DFM improved healing and augmented vascularity. Iron chelation by DFM administration has also shown that bone resorption is inhibited by limiting osteoclastic differentiation. It is to this end that a collagen based hydrogel material is ideal to, not only serve as a mimetic of an ECM, but to also serve as an aqueous solution in which to deliver hypoxia mimetic inducing compounds such as DFM.

The implementation of a hydrogel matrix also eliminates the issue of seeding efficiency of cells into the scaffold structure. When cells are added to the structure, they will be in a liquid suspension that will eventually become a solid hydrogel matrix. Because the proposed model will have a solid matrix, cells will not fall through the porous metal scaffold at the moment of seeding. Instead, they will remain suspended in the ECM-like gel.

Certain embodiments are directed to design a bone replacement implant capable of forming vascular structures in a hydrogel matrix, while allowing for osteoblast proliferation and cell differentiation. Osteoblasts can also successfully synthesize hydroxyapatite and retain their adhesion to the Ti-6Al-4V scaffold. The hydrogel matrix should contain all of the necessary supplements to favor angiogenesis and vascular structure maturation.

A hydrogel is a three dimensional network of polymer chains with water filling the void space between the macromolecules. In certain aspects the hydrogel includes a water soluble polymer that is crosslinked to prevent its dissolution in water. The water content of the hydrogel may range from 20-80%. In certain aspects the hydrogel may include natural or synthetic polymers. Examples of natural polymers include polyhyaluronic acid, alginate, polypeptide, collagen, elastin, polylactic acid, polyglycolic acid, chitin, and/or other suitable natural polymers and combinations thereof. Examples of synthetic polymers include polyethylene oxide, polyethylene glycol, polyvinyl alcohol, polyacrylic acid, polyacrylamide, poly(N-vinyl-2-pyrrolidone), polyurethane, polyacrylonitrile, and/or other suitable synthetic polymers and combinations thereof. For example, the hydrogel may include a crosslinked blend of polyvinyl alcohol (PVA) and poly(N-vinyl-2-pyrrolidone) (PVP). The hydrogel may also include beneficial additives that are released at the surgical site. For example, the hydrogel may include analgesics, antibiotics, growth factors, and/or other suitable additives.

### III. ANGIOGENESIS

The process of the development of new vasculature (angiogenesis) is one component of the wound healing process. Vascular structures serve as transport pathways for oxygen, nutrients and signaling molecules throughout the organismal systems. Because of the significance of this process, the capacity of implant to induce vascularization is essential to develop an ideal substitute of the original biological matter. It has been recently studied that cell-cell differentiation in developing organs is key for the development of angiogenesis. These interactions are mediated by Endothelial Cells (EC). It is these cells that form the first liner that becomes the basic template for the formation of veins and arteries. The main role of an endothelium is to serve as a transport pathway for oxygen. Therefore, ECs are equipped with oxygen sensor molecules such as Prolyl Hydroxylase Domain Enzymes (PHDs), and Hypoxia Inducible Factors-1α (Hif-1α). Despite the biological importance of vascular structure formation, achieving this remains a challenge in tissue engineering.

Angiogenesis can be initiated by certain growth factors, the most widely acknowledged signaling pathway being triggered by Vascular Endothelial Growth Factor (VEGF). Research has demonstrated that certain proteins, regulate the levels of VEGF secretion and play key roles in angiogenesis, the most important of these being the Hif-1α. Hif-1α acts as a transcription factor, translocating to the cell's nucleus under depravation of oxygen. This transcription factor increases the number of type HECs and osteoprogenitors through the process of hypoxia.

Hypoxia is defined as the deficiency of oxygen in tissues. When oxygen is depleted in tissue, a highly regulated process concerning cell survival becomes activated. Hif-1α is highly down-regulated by PHD-2 which target Hif-1α for degradation. During hypoxia, there is lack of oxygen in cells, which inactivates the prolyl hydroxylase domain proteins PHD1-3, which are oxygen-sensing. When this occurs, Hif-1α and Hif-2α proteins are no longer targeted for protein degradation and transcriptional responses are then activated to increase oxygen supply by angiogenesis through upregulation of VEGF. In general, Hif-1α promotes vessel sprouting, whereas Hif-2α mediates vascular maintenance. Hif-1α abrogation by siRNAs in HUVECs disrupts the formation of microcapillaries, but not Hif-2α. This is because Hif-2α does not stimulate the production of VEGF.

ECs migrate to reorganize themselves under hypoxia. The secretion of VEGF stimulates this reorganization. When VEGF is secreted, ECs also secrete metalloproteinases, whose role is to rearrange the ECM. After the ECM rearrangement, they begin to express CD44, allowing for an increased cell adhesion that enables the endothelium to maintain is microcapillar structure. Despite the high level of cellular organization to form microcapillaries, microvessel maturity is an issue as well. When microcapillaries form, endothelial cells may become quiescent (increased cellular half-life). However, the microsvascular structure may not always be retained, unless the endothelium recruits a pericyte liner. Pericytes are recruited by the endothelium when endothelial cell quiescence is achieved, which is determined by the secretion of Angiopoetin 1 & 2 (ANG-1 & ANG-2). The secretion of ANG-1 signals endothelium quiescence, whereas ANG-2 is secreted by Endothelial Tip Cells (ETCs). An ETC is a single endothelial cell randomly selected to commence the progression of a sprouting microvasculature. This process promotes vascular branching. In a physiological environment, ECs are held together by the Extracellular Matrix (ECM). This is a matrix represents a physical barrier that the endothelium can manipulate.

It has been previously reported that hypoxia induced by exposing cells in vitro and in vivo to CoCl₂ causes severe inflammatory response, resulting in the recruiting of macrophages. This has been observed in failed implanted structures that consist mainly of Cobalt-Molybdenum-Nickel alloys. In this particular research, particulate debris from the implanted alloy was analyzed against macrophages, resulting in hypoxia. The effects of cobalt ions on cells have been analyzed, but not the effects of hypoxia mimetic cellular responses with anything other than cobalt based materials. Despite these results, there have been a myriad of results demonstrating that hypoxic stress does not mediate cell death, instead, it promotes cell survival. It has been widely studied that Cobalt ions can stimulate the production of Reactive Oxygen Species (ROS), thus leading to mitochondrial insult, resulting in apoptosis. This leads to a controversial issue: does a hypoxia mimetic environment necessarily cause an undesirable response in wound healing?

### IV. HYPOXIA IN WOUND HEALING

Cells have evolved to respond to varied environments. Lack of free oxygen is one of them. Because oxygen is required for many cellular metabolic processes, such as the production of Adenosine Triphosphate (ATP), fatty acid synthesis and oxidative phosphorylation, cells are prepared to activate transcription factors that promote cell survival. Under a hypoxic response, the Hif-1α intracellular levels increase, as it is no longer targeted for degradation by PHD enzymes. Hif-1α can then dimerize with Hif-1β in the cell nucleus and initiate the transcription process that results in the expression of the VEGF gene. VEGF has been reported to promote an angiogenic response, and increase the activation of the Phosphatidyl Inositol-3-Kinase (PI3K)-Akt signaling pathway. It has been broadly researched and acknowledged that this particular signaling pathway is actively involved in the progression of tumor invasiveness and metastasis in a variety of cancer models. Hypoxia has been reported to increase the viability of cells and progression of survival signaling pathways. However, on a normal cell line, inhibiting the degradation of Hif-1α inhibits apoptosis, does not produce ROS (as Cobalt does), but results in promoting cellular differentiation and migration. Moreover, because the PI3K-Akt pathway becomes activated while a cell is experiencing a hypoxic response, therefore, diligent care must be taken in order to, not only select an appropriate hypoxic inducer, but to employ it at the correct concentrations. Despite the molecular signaling similarities between hypoxia stressed cells and cancer, the metabolic profiles of each are different. This suggests that, though the PI3K/AKT pathway is expressed, no adverse effects such as the immortalization of cells should be observed. The viability, proliferation, and population doublings of the cells exposed to various hypoxia inducing molecules must be addressed, and must not be limited to endothelial cells.

Deferoxamine Mesylate (DFM), also referred to as Deferoxamine (DFO) is an iron chelating agent; meaning that it binds to free iron ions in solution. This particular molecule is employed to regulate iron homeostasis in cells by chelating excess iron in solution. DFM is a well know inhibitor of PHD enzymes and has also been shown to increase bone density in osteoporosis mouse models. Despite there being other chemicals that may induce hypoxia in cells, i.e. CoCl₂, DFM has little known adverse effects.

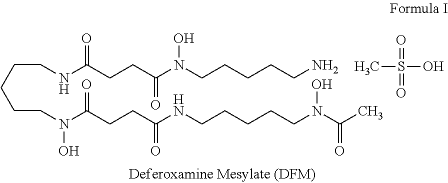

Because DFM binds to iron co-factors, the catalytic ability of PHD enzymes becomes hindered, leading to the stabilization of Hif-1α. DFM has been approved by the Food and Drug Administration (FDA) and is available for clinical use in the US. Currently, it is being used as an iron chelating agent to treat iron overdose from blood transfusions. As previously mentioned CoCl₂ triggers a hypoxic response and stabilized Hif-1α because it competes with iron for enzymatic active sites.

In recent years a wide number research papers have been published with promising applications for hypoxia in wound healing. These approaches include but are not limited to diabetic wound healing in fibroblasts, several mitochondrial related metabolic diseases such as Leigh Syndrome, and more recently to treat brain hemorrhage. The biomedical applications of hypoxia can be tailored to combat a variety of wound healing situations. It has also been recently reported that inhibiting PHD2 enzymes and stabilizing Hif-1 a increases the survival rate of newly implanted cells in bone. It has been reported that the levels of ROS species in endothelial cells decreases, enabling cells to undergo redox homeostasis and glycogen storage. This further suggests that a hypoxia mimetic, but not hypoxia as a lack of oxygen maintains the integrity of cellular metabolism by stabilizing Glutathione S Transferase (GST). Because of the ever increasing evidence that hypoxia can support regenerative medicine, in this research, a hypoxia mimetic will be applied to promote vascularization, pre-osteoblast differentiation and wound healing for newly implanted bone replacement implants.

### V. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

A. Materials & Methods

Materials. Type-A gelatin (Cat. No. 901771) was procured from MP Biomedicals, France. Alginate (alginic acid sodium salt from Brown Algae, Cat. No. A0682), N-hydroxy-succinimide (NHS, Cat. No. 130672), and 1-ethyl-3-(3-dimethylaminopropyl) carbodiimide (EDC, Cat. No. 22980) were obtained from Sigma-Aldrich, USA. Nanocrystalline hydroxyapatite was synthesized in-house using suspension-precipitation method using precursors: calcium oxide (CaO) and orthophosphoric acid (H₃PO₄).

Synthesis of nanocrystalline hydroxyapatite (nHA). Nanocrystalline hydroxyapatite powder was synthesized using suspension-precipitation approach involving reaction between CaO and H₃PO₄ (Tagai and Aoki, Preparation of synthetic hydroxyapatite and sintering of apatite ceramics: John Wiley and Sons, 1980). In this approach, a solution of CaO was first prepared in high purity deionized water (19.6 g/l) and the solution temperature maintained at 80° C. during the reaction. The solution was titrated with 0.15 M H₃PO₄ acid. A 0.15M solution of H₃PO₄ was prepared by adding 9.5 ml H₃PO₄ in 1 L of deionized water. The pH of the solution was adjusted to ˜12 using ammonium hydroxide solution (NH₄OH). After completion of reaction, the solution was filtered and precipitate (nanocrystalline hydroxyapatite) was collected, followed by drying at 80° C. for 24 h in an electric oven. HA powder was calcined at 800° C. for 2 h in air using muffle furnace. The calcined powder was ball milled for 16 h at 200 rpm using agate milling media (Pulveristte 7 premium line, Fritsch, Germany). The ball to powder ratio was 4:1. Toluene was added during milling to avoid the sticking of powder (Kumar et al., Journal of Biomedical Materials Research Part B: Applied Biomaterials. 2013, 101B:223-36). Ball milled powder was characterized by the X-ray diffraction (Bruker's D8 Discover, Germany). The XRD data was analyzed and compared with hydroxyapatite standard (pdf no. 74-0566, International Committee for Diffraction Data). Furthermore, transmission electron microscopy (TEM) and selected area diffraction pattern (SAED) were used to characterize the particle size and morphology of ball-milled nHA particles (Kumar et al., Acta Materialia 2013, 61:5198-215).

Synthesis of hybrid injectable pre-hydrogel reinforced with nHA. A pre-hydrogel was synthesized, followed by final cross-linking with CaCl₂. It may be noted that to synthesize pre-hydrogel, CaCl₂ was not used for cross-linking during gel synthesis. It was only after cell encapsulation (cell loading in pre-hydrogel), that final cross-linking was carried out using 0.05 M CaCl₂ to further enhance the strength. Four compositions were prepared and based on preliminary study of osteoblast cell culture, composition A was selected (Table 1). To synthesize 100 ml pre-hydrogel, an aqueous solution of nanocrystalline hydroxyapatite (nHA) was prepared by mixing 500 mg nHA powder in 100 ml high purity deionized water and solution was kept on magnetic stirrer (1000 rpm) at 40° C. After 15 min of mixing, 2% gelatin (20 mg/ml of nHA suspension) and 2% alginate (20 mg/ml of nHA suspension) were added and stirred at 40° C. for another 15 min. Furthermore, 250 mg EDC and 150 mg NHS were added and stirred at 40° C. for 24 h. The prepared pre-hydrogel reinforced with nHA was filled in a sterilized syringe and kept under UV for 12 h for sterilization. After sterilization, the pre-hydrogel was loaded with cells and cross-linked with 0.05 M CaCl₂ for 5, 10, or 15 min. Pre-hydrogel without cells, cross-linked with 0.05 M CaCl₂ for 5, 10, or 15 min were considered as controls. Next, the prepared hydrogels (cross-linked with 0.05 M CaCl₂) were kept in 1× PBS for 15 min to remove excess CaCl₂ and then transferred to culture medium and incubated for 5 days in 5% CO₂ and 95% humidity at 37° C. in a sterilized environment. After incubation, cell-loaded hydrogels were characterized for cell viability and proliferation, while hydrogels without cells were characterized for phase assemblage and porous structure. The prepared hydrogel (loaded with cells) was infiltrated in to Ti-6Al-4V scaffolds of 2 mm mesh size (55% porosity and 300 μm pore size). The dimensions of sample were 8 mm×8 mm×2 mm. Prior to this, the scaffold surface was polished using 0.25 μm alumina suspension, followed by ultrasonication of scaffolds in distilled water, acetone, and 70% ethanol for a total duration of 180 min.

Phase assemblage and pore architecture. To determine the phase assemblage and pore architecture, the hydrogel (cross-linked with 0.05 M CaCl₂ for 10 min) was studied by X-ray diffraction (XRD), Fourier transform-infrared spectroscopy (FT-IR), and scanning electron microscope (SEM) in secondary electron (SE) mode. For SEM, samples were coated with gold to avoid charging during imaging. For SEM, XRD, and FT-IR, the hydrogel sample was kept at −80° C. for 12 h, followed by freeze drying for 12 h to get a dried porous scaffold. To characterize by XRD and FT-IR, the scaffolds were ground in agate mortar and pestle to make fine powder. In the case of FT-IR (FT/IR-4600LE, Jasco, Japan), freeze dried hydrogel powder sample was mixed with KBr in the ratio of 1:200, and a thin pellet of 3 mm diameter was made. The absorption of IR radiation was recorded from 4000 to 600 cm⁻¹ XRD (D8 Discover, Bruker's diffractometer, Germany) was carried out at 40 kV voltage and 40 mA current using CuKα wavelength (1.54056 Å) from (2θ) 20° to 90° at a scanning rate of 2°/min and with an increment (step size) of 0.02°.

Effect of cross-linking time on the dissolution behavior of hydrogel. To determine the stability and integrity of the hydrogels, dissolution study in cell culture media was carried out for 2, 4, 6, and 8 days. In this regard, 8 well plate (Cat. No. 267062, Thermo Scientific Nunc, USA) was used as a mold to obtain a hydrogel sample of 2 mm thickness. In each well, 4 ml pre-hydrogel was added, followed by cross-linking with 0.05 CaCl₂ for 5 (category I), 10 (category II), and 15 min (category III). Cross-linked samples were washed with 1× PBS, followed by immersion of samples in 1× PBS for 15 min to ensure the removal of excess CaCl₂. The molded hydrogel samples were cut in to dimension of 8 mm×8 mm×2 mm. Next, the samples were transferred to 24 well plate and well plate was sealed with parafilm (Cat. No. PM-996) to avoid the loss of water during the test period, followed by UV sterilization for overnight. After sterilization, samples were immersed in equal volume (2 ml) of 1× PBS and well plates were sealed again by parafilm and kept in a hot air oven at 37° C. for 2, 4, 6, and 8 days. Experiments were carried out on triplicates and repeated for at least three times to obtain statistically relevant data. After 2, 4, 6, and 8 days of incubation, solution from each well was carefully removed and absorption was measured at 210 nm. 1xPBS was used as a reference.

To estimate the amount of dissolved hydrogel, a standard curve was plotted using different amount of alginate in 1× PBS in the range of 0.05 mg/ml to 5 mg/ml and optical density of solution was measured at 210 nm. A curve between dissolved amount of alginate and optical density was plotted using Excel (Office 2013, Microsoft, USA). Corresponding to the plotted curve, a trend line was drawn to estimate the equation using curve trend and slope. This equation was used to estimate the amount of dissolved alginate from hydrogel during dissolution. Since, both gelatin and alginate indicate absorption at 210 nm, thus, obtained OD corresponded to the total amount of dissolved material (sum of alginate and gelatin) from hydrogel. Thus, dissolved amount of alginate was half of the total dissolved hydrogel.

Study of sorption kinetics of monocrystalline hydroxyapatite reinforced hydrogel (nanocomposite) in PBS. To study sorption kinetics, nanocrystalline hydroxyapatite reinforced hydrogel (nanocomposite) was filled in 8 well plate (4 ml in each well) and 0.05 M CaCl₂ was added for 10 min for cross-linking. Next, cross-linked hydrogel was kept in 1× PBS for 15 min to remove excess CaCl₂. The nanocomposite was cut in a rectangular shape of dimensions 30 mm×20 mm×4 mm and refrigerated at −80° C. for 5 h, followed by freeze drying for overnight. The dried samples were weighed and immersed in 1× PBS at 37° C. The swollen nanocomposite samples were removed from 1× PBS after 30 min and excess surface water was removed using filter paper, and weighed. After this, the samples were again immersed in a fresh 1× PBS at 37° C. This process was repeated until equilibrium swelling was reached. The change in weight during this process was recorded as a function of time. All the measurements were carried out in triplicate to obtain statistically relevant data. Using these data, swelling ratio, equilibrium swelling ratio, swelling rate, and equilibrium water content were calculated using following equations:

$$\begin{matrix}Swelling ratio = \frac{{m}_{t} - {m}_{d}}{{m}_{d}} = degree of swelling & ( 1 ) \\ Equilibrium swelling ratio = \frac{{m}_{equ} - {m}_{d}}{{m}_{d}} & ( 2 ) \\ Swelling rate = \frac{{m}_{t + Δ t} - {m}_{t}}{Δ t} = change swelling content per unit time & ( 3 ) \\ Percentage equilibrium water content = \frac{{m}_{equ} - {m}_{d}}{{m}_{equ}} \times 100 & ( 4 )\end{matrix}$$

where, m_{d}, mₜ, and m_{equ} are the weight of the dried nanocomposite, weight of the swollen nanocomposite at time ‘t’, and weight of the swollen nanocomposite at equilibrium state, respectively.

PBS desorption kinetics for swelled nanocomposite. Samples were removed from 1× PBS after attaining swelling equilibrium. The excess water from the samples surface was removed using a filter paper. Next, the nanocomposite samples were weighed. This weight is considered as swollen weight (w_{∞}). Following this, the samples were kept at 37° C. with constant humidity for 30 min. The process was repeated until the samples were completely dried and a constant weight was obtained. The amount of PBS desorption from nanocomposite was documented as a function of time and PBS desorption was calculated using following equation:

$$\frac{{M}_{t}}{{M}_{∞}} = \frac{{w}_{t} - {w}_{0}}{{w}_{∞} - {w}_{0}} = PBS desorption ratio \qquad ( 5 )$$

Where, wt, w₉, and w_{∞} are the weight of nanocomposite at time ‘t’, initial time ‘0’, and completely dried time ‘∞’, respectively.

Cytocompatibility assessment. Alginate-gelatin hydrogel reinforced with nanocrystalline hydroxyapatite (nanocomposite), loaded with MC3T3-E1 pre-osteoblast cells (cell density ˜10⁶ cells/ml of hydrogel) was infiltrated in Ti-6Al-4V scaffolds, followed by cross-linking with 0.05 M CaCl₂ for 5, 10, and 20 minutes. These samples with 3D-porous architecture (Ti-6Al-4V) infiltrated with hydrogel (comprised of cells and nanocrystalline hydroxyapatite) are referred as hybrid nanocomposite. These hybrid nanocomposites were incubated in complete culture medium for 5 days, followed by studies involving live/dead assay, cell morphology (expression of actin, vinculin and staining of nucleus), and MTT assay (to study metabolically active cells and thus measure the cell proliferation).

Live/dead assay. Pre-hydrogel samples (nHA reinforced hydrogel and loaded with osteoblasts, infiltrated in Ti-6Al-4V scaffold) were subjected to cross-linking by 0.05 M CaCl₂ for 5, 10, and 15 min. Following the cell culture protocol described above, after 6 days of incubation, samples were analyzed using live/dead assay to study the effect of cross-linking time on cell viability and to select appropriate samples for further study based on cell viability and cross-linking time data. The details of live/dead assay are reported elsewhere (Kumar et al., Journal of biomaterials applications 2016, 0885328216658376). Briefly, staining agent for live cells (live green) and dead cells (dead red) (Cat. No. R37601, Live/Dead imaging kit, Life Technologies, USA) was used to make the stock solution. Next, the samples were washed with 1× PBS and each sample was treated with equal volume of stock solution. The samples were incubated for 15 min at room temperature (20-25° C.), stored at 6° C., and studied using fluorescence microscopy within 2 h. Green and red colors in the micrograph denoted live and dead cells, respectively. On the basis of these results and stability of hydrogel in culture medium, 10 min cross-linking time was considered optimal for further studies. Thus, further studies were carried out on the samples cross-linked with 0.05 M CaCl₂ for 10 minutes (category II).

MTT assay/cell proliferation assay. For MTT assay, samples were prepared by the addition of 1 ml pre-hydrogel in each well of 24 well plate, followed by cross-linking with 0.05 M CaCl₂ for 10 min. The cross-linked samples were washed with 1× PBS for 15 min and kept under UV for overnight. To avoid the water sorption, well plate was sealed with parafilm. After UV sterilization, samples were again washed with 1× PBS, followed by a treatment with 1 ml complete culture medium for 1 h. After 1 h, media was removed and MC3T3-E1 cells with a cell density of 50,000 cells/ml were seeded on the hydrogel surface. After 4 h of incubation, 1 ml complete culture media was added in each well to maintain total 2 ml solution in each well. Cells seeded on hydrogel surface were incubated for 2, 4, 6, and 8 days. During the incubation period, old media was replaced with new media on each day. To evaluate cell viability and cell proliferation, MTT (3(4, 5-dimethylthiazol-2-yl)-2, 5-diphenyl tetrazolium bromide)) assay was used after 2, 4, 6, and 8 days of incubation of samples of category II. The details of MTT protocol has been reported elsewhere (Kumar et al., Materials Science and Engineering C 2012, 32:464-9). Briefly, after pre-determined incubation period, culture media was replaced with 10% MTT reagent (in 1× PBS), followed by incubation of samples in CO₂ incubator for 6 h. This resulted in reduction of MTT salt into insoluble formazan crystals. After incubation, MTT reagent was removed carefully and violet colored formazan crystals were dissolved using DMSO (dimethyl sulfoxide, D8418, Sigma Aldrich, France). The violet color solution was transferred to a 96 well plate to measure the optical density of solution at 570 nm wavelength using ELISA (enzyme-linked immunosorbent assay) plate reader (ELx800, BioTek, USA). The data obtained was normalized by the optical density of DMSO solution. Furthermore, obtained optical density was normalized with the amount of formazan crystals absorbed in to hydrogel due to porous nature of the hydrogel. For this, hydrogel was transferred to 50 ml centrifuge tube and diluted 5 times. Next, probe sonication was used to dissolve the hydrogel. Now, the dissolved hydrogel with formazan crystals was centrifuged for 10 minutes at 10,000 rpm. The 200 μl of supernatant was transferred to 96 well plate and optical density was measured at 570 nm. The obtained optical density was multiplied by 5 to equalize the dilution factor. This value of optical density was added to the original value of optical density to obtain the final value of optical density.

Cell-cell and cell-material interactions. An actin cytoskeleton and focal adhesion staining kit (cat. No. FAK100, Millipore, USA) was used to study the cell-cell and cell-material interaction, after 2 and 6 days of incubation, the samples were washed twice with 1× PBS. To fix the cells, the samples were kept in 4% formaldehyde at room temperature for 20 min. Next, samples were washed twice with 1× PBS. These samples were treated with 0.1% tritonx 100 for 6 min. This resulted in cell wall permeabilization. To reduce the non-specific binding of staining agents, after washing the samples with 1× PBS twice, samples were further treated with 5% FBS for 30 min, followed by washing twice with 1× PBS. These samples were stained for 60 min by a staining agent (green color, specific for focal adhesion contact points of cells) to study the expression of vinculin. Furthermore, after washing twice with 1× PBS, the samples were stained for 60 min with a staining agent (red color), specific for actin stress fibers to study the reorganization of cytoskeleton. After washing twice with 1× PBS, cell nucleus was stained with DAPI (blue color) for 10 min. The stained samples were washed three times with 1× PBS and stored at 6° C. in 1× PBS in dark until imaging by florescence microscope.

Alkaline phosphatase (ALP) assay. An APL assay kit (cat. No. DALP-250, BioAssay Systems, USA) was used to study the efficacy of bone formation of the designed nanocomposite hydrogel, hydrogel samples crosslinked for 10 min, followed by culture of osteoblasts (50,000 Cells/ml) for 4 days, and then differentiation for 6, 12, and 18 days were selected for ALP assay. In an alkaline environment, ALP catalyzes the hydrolysis of phosphate esters. In this method, ALP present in the solution (due to differentiation of osteoblast on the biomaterial surface) hydrolyzes the p-nitrophenyl phosphate (pNPP) in to p-nitrophenol and phosphate. This yellow colored product shows maximum absorbance at 405 nm. Since, ALP enzyme is present in bone and the rate of hydrolysis is directly proportional to the activity of ALP, therefore, recorded OD using ELISA plate reader can be correlated with the bone formation ability of the biomaterial. Briefly, protocol comprises of preparation of working solution, cell lysis, followed by optical density measurement at 405 nm. The ‘working solution’ was prepared by mixing 5 μl magnesium acetate and 2 μl pNPP in to 200 μl assay buffer. Solution was stored at 4° C. At this temperature, solution can be stored for not more than 48 h. Next step, sample was washed with 1× PBS and 500 μl of 0.2% Triton-X100 (in distilled water) was added on each sample. The samples were incubated for 30 min at room temperature to lysis the cells. This lysed cell solution in Triton-X is designated as ‘sample solution’. Next 50 μl of sample solution and 150 μl of working solution was mixed in a centrifuge tube and transferred to 96 well plate to measure the absorption at 405 nm at time 0 and 5 min. Further, 200 μl of calibration solution was added in another 96 well plate and absorption was recorded at 405 nm. In a similar way, 200 μl of distilled water was added in another 96 well plate and absorption was recorded at 405 nm. Using these values, ALP activity of the sample can be calculated using following equation.

$$= \frac{( {OD}_{sample at time t} - {OD}_{sample at time 0} ) \times reaction volume}{t \times ɛ \times l \times sample volume} mmol \text{/} L \cdot min \qquad ( 6 )$$

Where, t is the incubation time (min), ϵ is extinction coefficient (molar absorption coefficient) of ρ-nitrophenol (=18.75 mmol⁻¹·cm⁻¹), l is the light path (cm) and for 96 well plate is equal to (OD_{calibrator}−OD_{distilled water})/ϵ·c; where, c is the concentration of calibrator.

After substituting the values of ϵ, equation 6 becomes,

$$ALP activity of sample == \frac{( {OD}_{sample at time t} - {OD}_{sample at time 0} ) \times reaction volume}{t \times ( {OD}_{calibrator} - {OD}_{distilled water} ) \times sample volume} \times 35.3 µmol \text{/} L \cdot min \qquad ( 7 )$$

Statistical Analysis. The data obtained was analyzed using a statistical analysis software (SPSS 19.0, IBM, USA). Post-hoc tests (multivariate comparison) was used to compare the mean values of samples. Two way ANOVA (Analysis of Variance) was used with Dunnett t (represented by symbol *) and Dunnett C (represented by symbol **) tests to estimate the significant difference between the samples mean and in comparison with control at p<0.05, respectively (Yuksel et al., International Journal of Pharmaceutics, 2000, 209:57-67). All data presented as mean±standard error of mean using Origin software (version 8.5, Origin Lab Corporation, USA).

B. Results

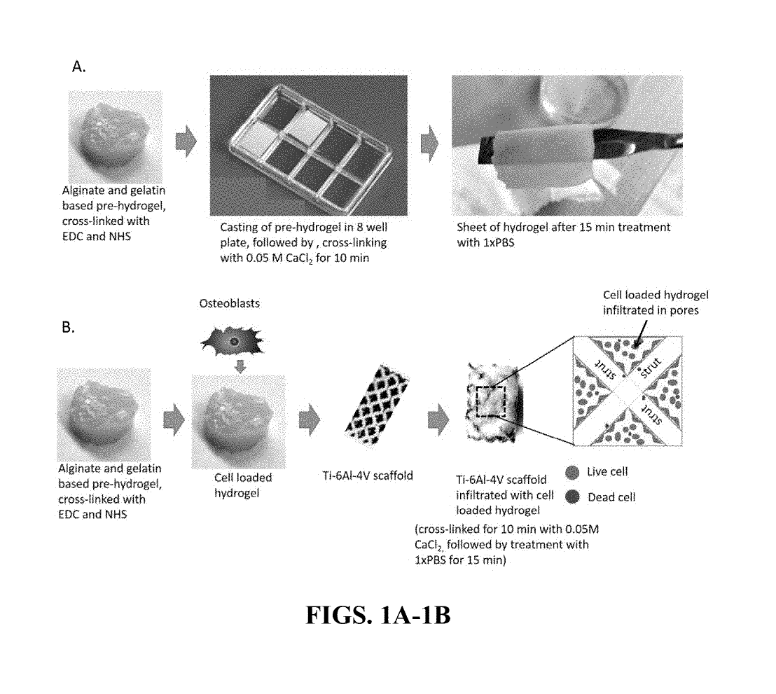

Synthesis of hydrogel. First, nanocrystalline hydroxyapatite was synthesized in lab using suspension-precipitation method, prior to synthesis of alginate and gelatin based hydrogel, reinforced with nanocrystalline hydroxyapatite (FIG. 1). Scanning electron microscopy (SEM) and transmission electron microscopy (TEM) study confirmed the monolithic phase of hydroxyapatite with nano-sized needle shape particles of ˜80 nm length and ˜30 nm diameter (Kumar et al., Acta Materialia, 2013, 61:5198-215). The hydroxyapatite prepared by this method was highly bioactive and cytocompatible (Kumar et al., Journal of Biomedical Materials Research Part B: Applied Biomaterials, 2013, 101B:223-36; Kumar et al., Journal of Biomedical Materials Research Part A, 2013, 101:2925-38; Kumar et al., Journal of biomaterials applications, 2016, 30:1505-16).

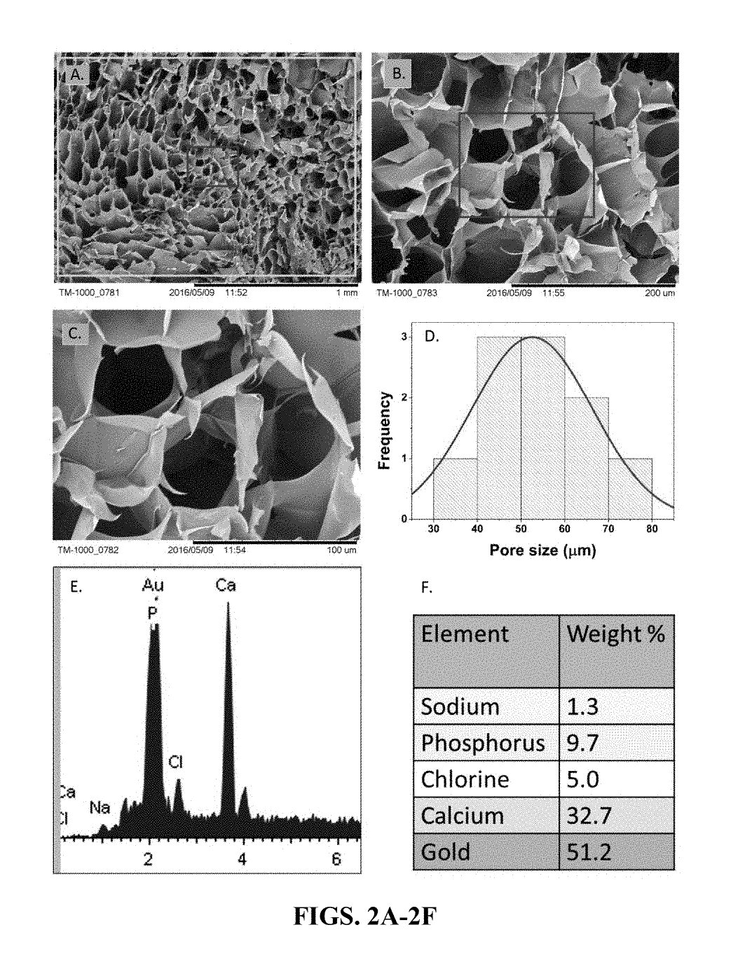

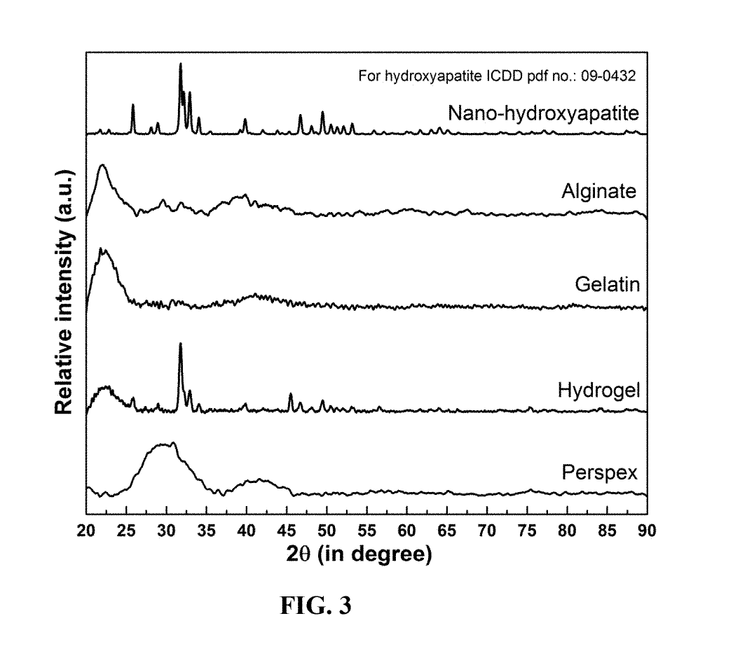

Microstructure and phase assemblage. The scanning electron micrographs of freeze-dried hydrogel after final cross-linking with CaCl₂ for 10 min revealed highly porous structure with ≤2 μm wall thickness of the pores (FIG. 2a, 2b, 2c, 2d). As shown in FIG. 2e, 2f presence of hydroxyapatite (by measuring the calcium and phosphorous) and sodium alginate (by measuring the sodium) was confirmed by Energy Dispersive Spectroscopy (EDS). Presence of chlorine and gold is associated with CaCl₂ (used for the cross-linking) and gold coating (to minimize the charging during SEM). XRD analysis revealed the presence of diffraction peaks corresponding to crystalline hydroxyapatite in nano-hydroxyapatite powder as well as in hydrogel (FIG. 3). The presence of alginate and gelatin in hydrogel was also confirmed by XRD. This was confirmed by comparing the X-ray diffraction peak position and intensity with ICDD (international committee for diffraction data) standard of monolithic hydroxyapatite (pdf #09-0432). During XRD experiment, perspex was used as a substrate for the powder samples. Thus, to avoid any misinterpretation, XRD of perspex was obtained for comparison. The sharp and narrow peaks of hydroxyapatite confirmed the crystallinity of the synthesized hydroxyapatite particles. There was no significant change in the hydroxyapatite diffraction peaks in the hydrogel.

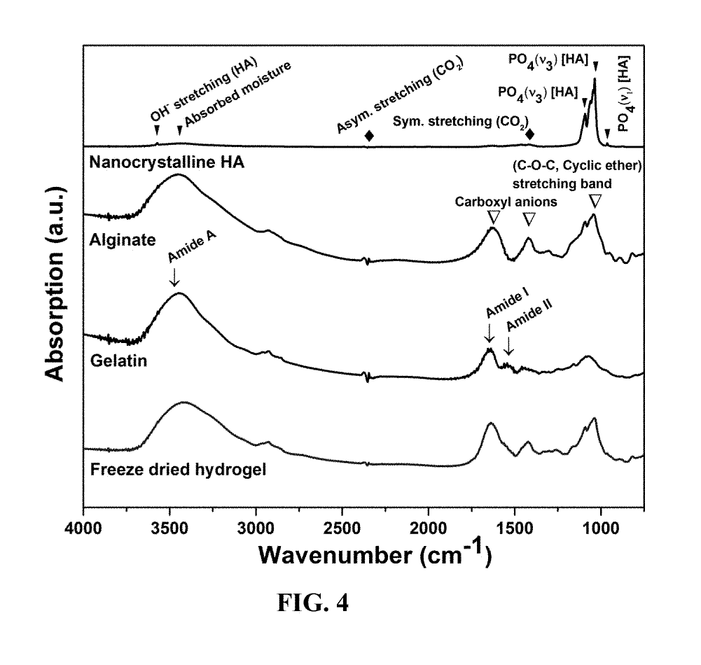

In XRD data, it is very difficult to identify the presence of alginate and gelatin because of high intensity peaks of hydroxyapatite in comparison to alginate and gelatin. Therefore, FT-IR was used as a complimentary tool to identify the presence of alginate and gelatin in the hydrogel (FIG. 4). Presence of FT-IR peaks corresponding to OH stretching (3574 cm⁻¹, corresponds to OH group in hydroxyapatite) as well as v₁(969 cm⁻¹) and v₃(1038 and 1098 cm⁻¹) of PO₄ confirmed the presence of hydroxyapatite phase in the powder sample. The peaks corresponding to CO₂ was environmental CO₂ impurity, trapped in sample during KBr pellet fabrication for the FT-IR. Next, the presence of peaks at 3450 cm⁻¹, 1618 cm⁻¹, 1440 cm⁻¹, and 1050 cm⁻¹ corresponded —OH group, —COO—, —COO—, and C—O, respectively. This confirmed the presence of alginate in the starting powder sample. Furthermore, the presence of FT-IR bands at 3351 cm⁻¹, 2948 cm⁻¹, 1658 cm⁻¹, and 1551 cm⁻¹ corresponded to amide-A, amide-B, amide-I, and amide-II, respectively confirming the gelatin phase. In hydrogel, the presence of signatory absorption peaks of hydroxyapatite, alginate, and gelatin in hydrogel confirmed the presence of these materials in hydrogel without any signature of conformational change in the structure of alginate and gelatin.

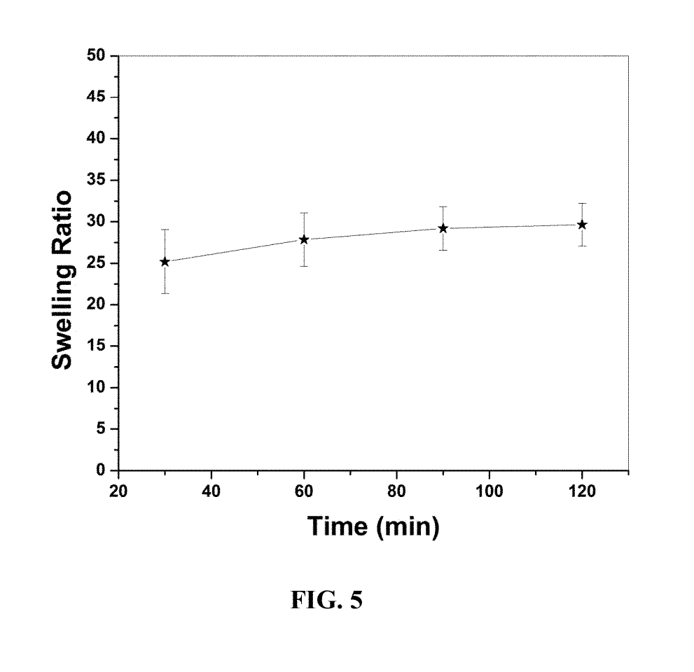

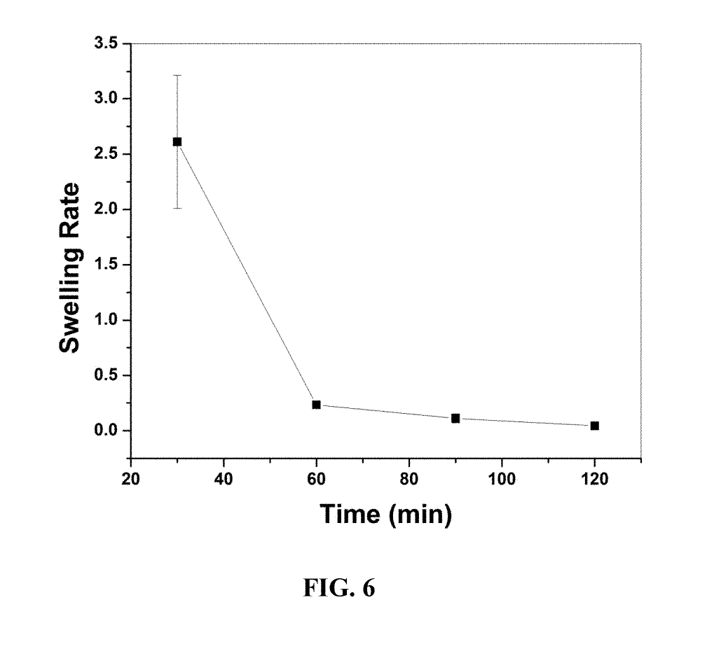

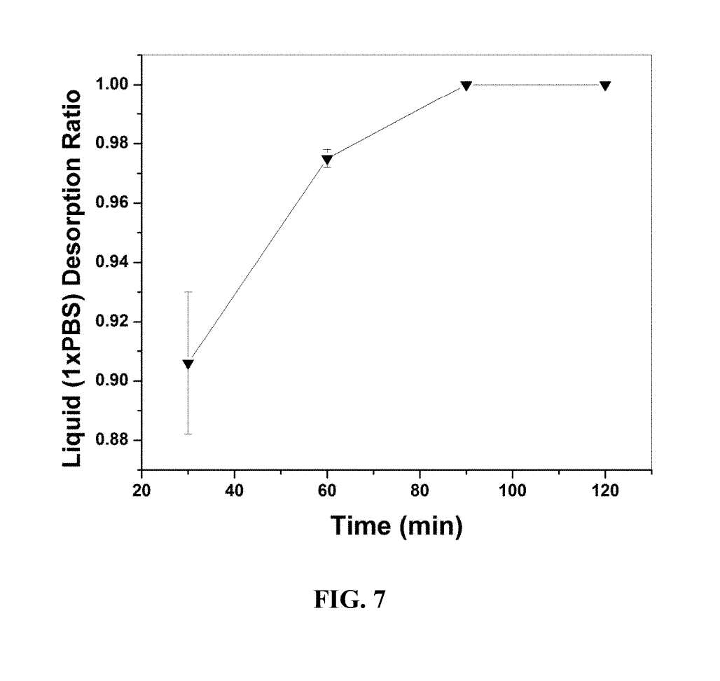

Sorption and desorption kinetics. Sorption and desorption kinetics of synthesized hydrogel was studied by measuring the swelling ratio (FIG. 5), swelling rate (FIG. 6), and desorption ratio of hydrogel (FIG. 7). The sorption kinetic study was carried out to estimate the water absorption capability of the hydrogel. The equilibrium swelling ratio and percentage equilibrium water (1× PBS) content were 29.64±3.16 and 96.67±0.33, respectively. Furthermore, a study from 30 min to 120 min showed a stable swelling ratio with time. However, after immersion of hydrogel in 1× PBS, a rapid decrease in swelling rate was noted from 30 min to 60 min. The swelling rate decreased from 60 min to 90 min and then became stable.

On drying of hydrogel at 37° C. in hot air oven, an increase in desorption ratio of 1× PBS was noted from 30 to 90 min. Beyond 90 min incubation, the desorption ratio was stable with time.

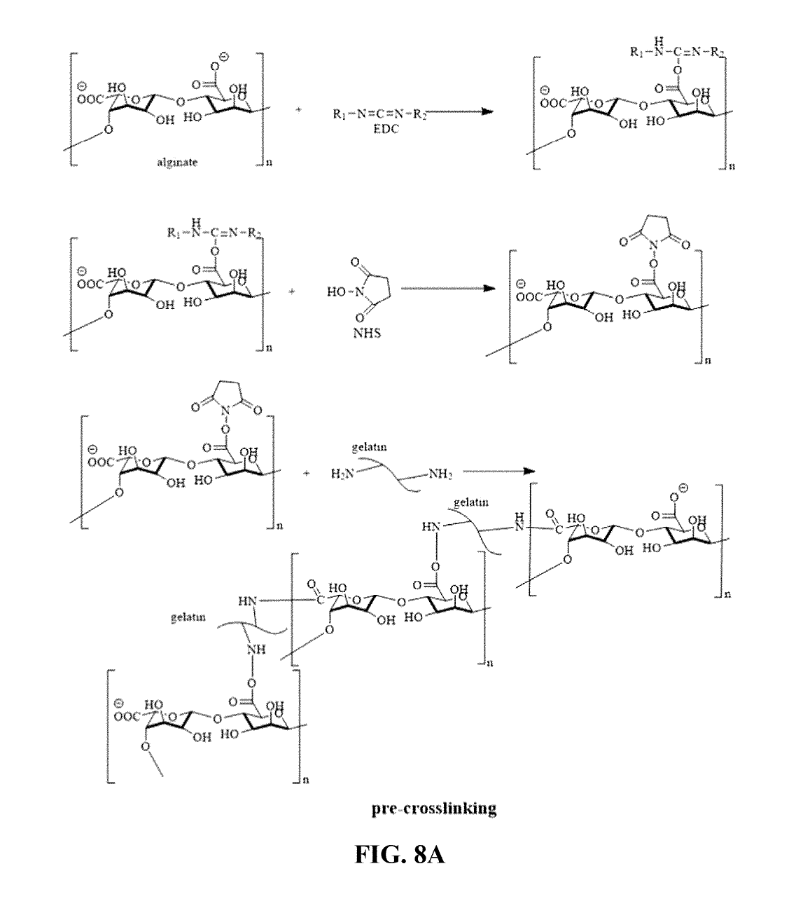

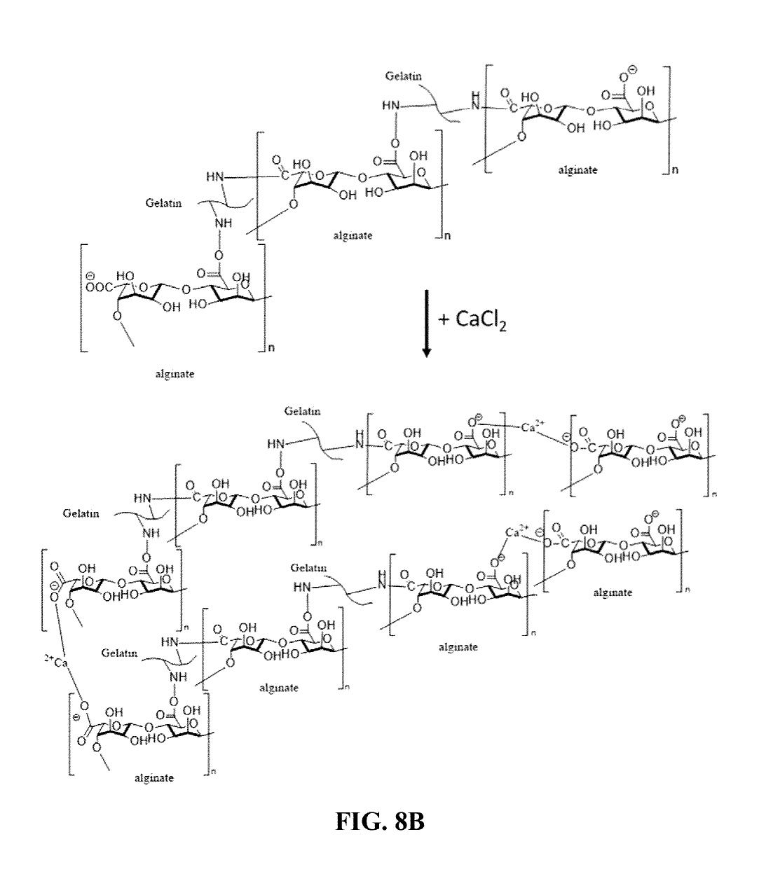

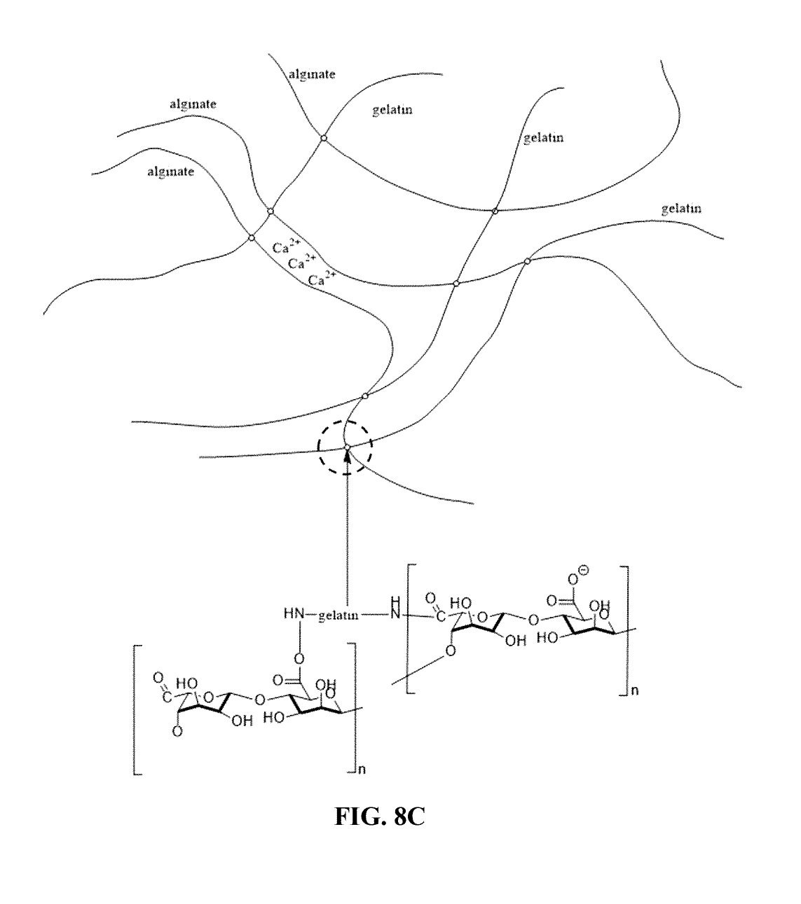

Dissolution study in 1× PBS. To study the effect of cross-linking time on the dissolution behavior of hydrogel samples, equal sized pre-hydrogel samples were cross-linked for three different time scale (5, 10, or 15 min) and kept in 24 well plate in 1× PBS at 37° C. for 2, 4, 6, and 8 days, with one sample in one well and 2 ml 1× PBS in each well. After 2, 4, 6, and 8 days of immersion, solution containing the dissolved hydrogel was removed carefully from well plate without disturbing the integrity of the hydrogel samples and diluted with MQ water, followed by measuring the absorption at 210 nm. The amount of dissolved hydrogel was calculated using a standard plot between amount of dissolved material vs. absorption at 210 nm. It is important to mention that both gelatin and alginate indicated absorption peak at 210 nm. Thus, the observed absorption value was directly proportional to the sum of absorption of radiation (λ=210 nm) by alginate and gelatin. Thus, the amount of dissolved alginate or gelatin was equal to half of total absorption value. FIG. 8 shows the reactions among the hydrogel components. As shown in FIG. 8a, first EDC bonded to the carboxyl group of alginate and then NHS bonded to the alginate by replacing EDC. Furthermore, this reaction product bonded together through gelatin to make the precursor for the hydrogel, and named as pre-hydrogel. In the next step, as shown in FIG. 8b, this pre-hydrogel was cross-linked with CaCl₂ to obtain the hydrogel. In FIG. 8c, a summary of all the gelation process used in the hydrogel synthesis is presented.

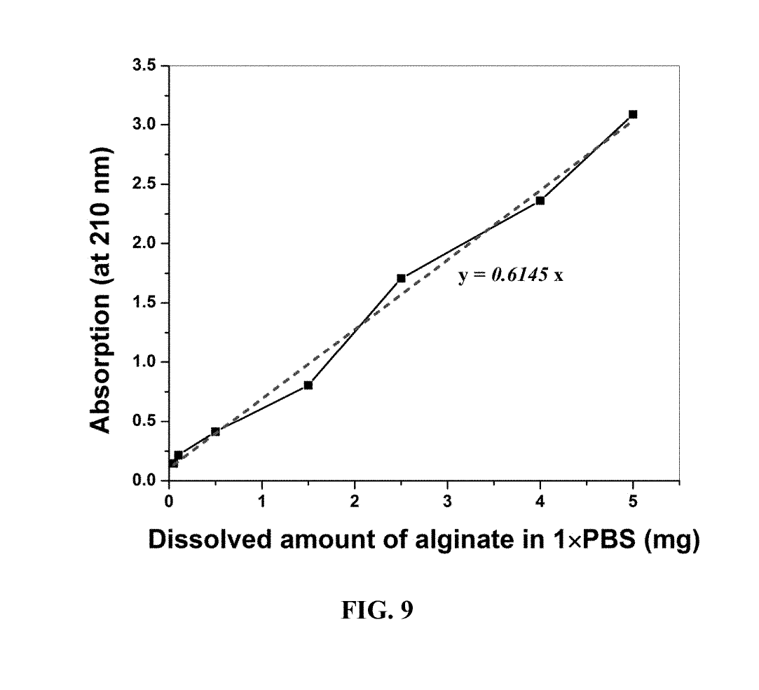

The standard plot was used to calculate the dissolved amount of alginate. For this, different amount of alginate (0.05, 0.1, 0.5, 1.5, 2.5, 4, and 5 mg) was dissolved in 2 ml 1× PBS. The corresponding absorption at 210 nm was 0.145, 0.215, 0.412, 0.804, 1.706, 2.361, and 3.086. FIG. 9 shows the plot between dissolved amount of alginate and absorption at 210 nm. A trend line drawn on the plot shows a near linear relationship between dissolved alginate and absorption. A formula (absorption=0.6145×dissolved amount of alginate) corresponding to trend line was calculated using Microsoft Excel. This formula was used to calculate the dissolved amount of hydrogel in 1× PBS at 37° C.

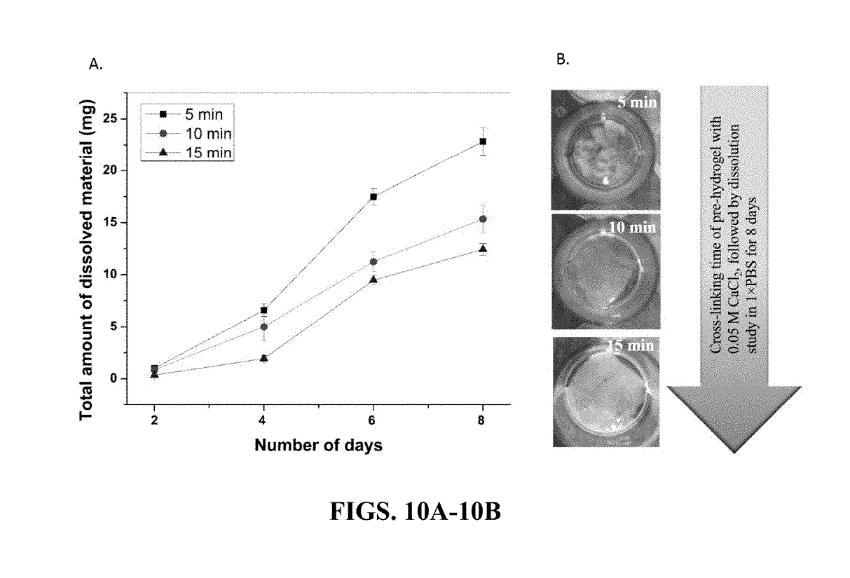

FIG. 10 shows the dissolution profile of hydrogel in 1× PBS, immersed for 2, 4, 6, and 8 days at 37° C. During dissolution period the media was not replaced. To avoid the loss of water due to evaporation and to maintain humidity, the well plate was sealed with parafilm. Results showed a significant effect of cross-linking time on the dissolution behavior with highest dissolution recorded in samples cross-linked for 5 min. However, samples cross-linked for 15 min indicated lowest dissolution. Cross-linking for 10 min led to moderate dissolution. Importantly, samples cross-linked for 10 min showed a linear relationship between number of days of immersion of hydrogel in 1× PBS with burst dissolution profile. In contrast to this, samples with 5 min and 15 min cross-linking time show a burst dissolution after 4^{th} day of immersion. However, the rate of dissolution was low in the case of 15 min cross-linked samples than samples cross-linked for 5 min. Digital photographs of samples, captured after 8 days of immersion also confirmed the effect of cross-linking time on the dissolution with complete dissolution of 5 min cross-linked samples as compared to samples cross-liked for 10 and 15 min (FIG. 10).

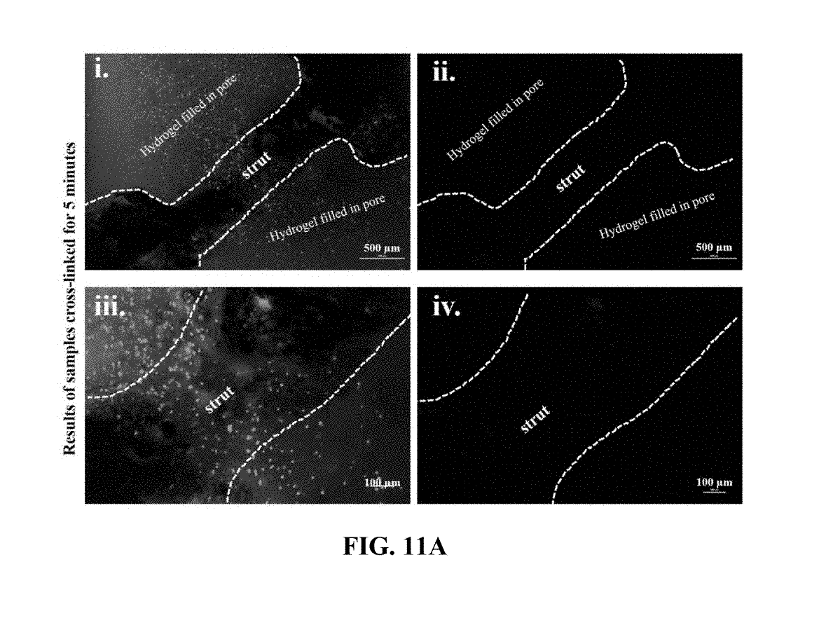

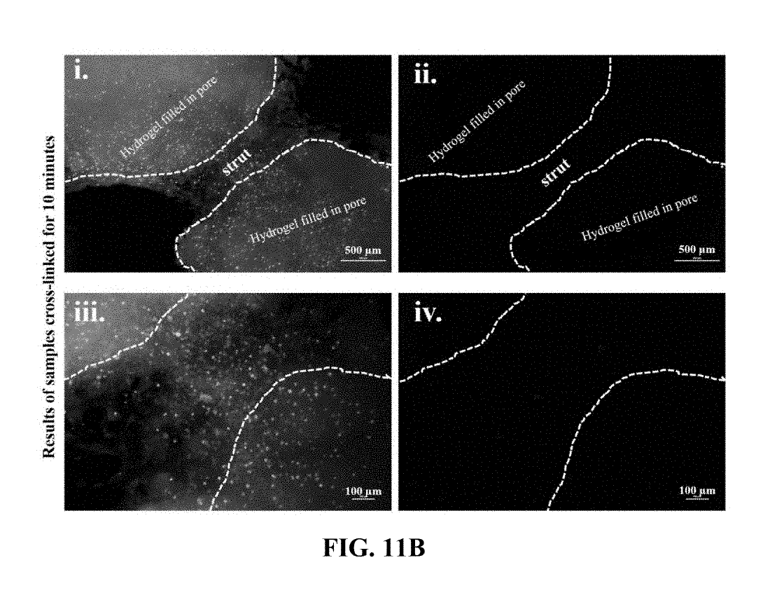

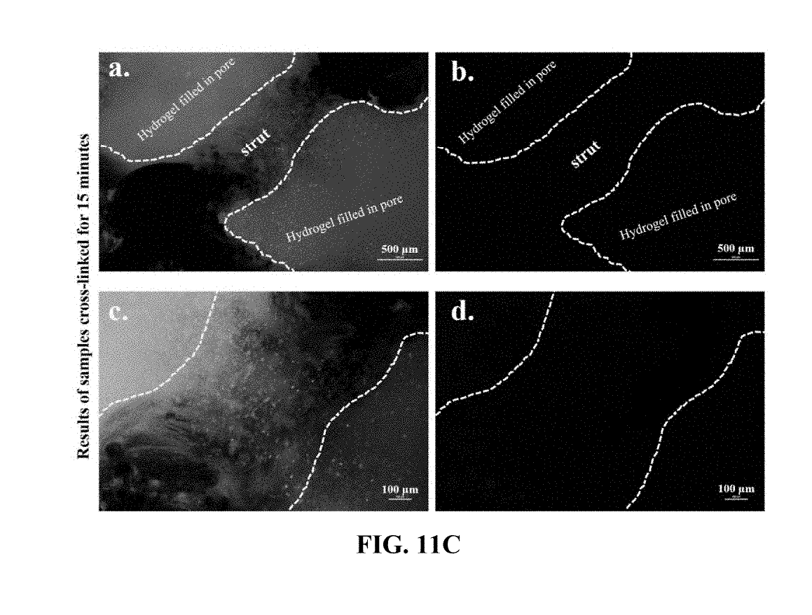

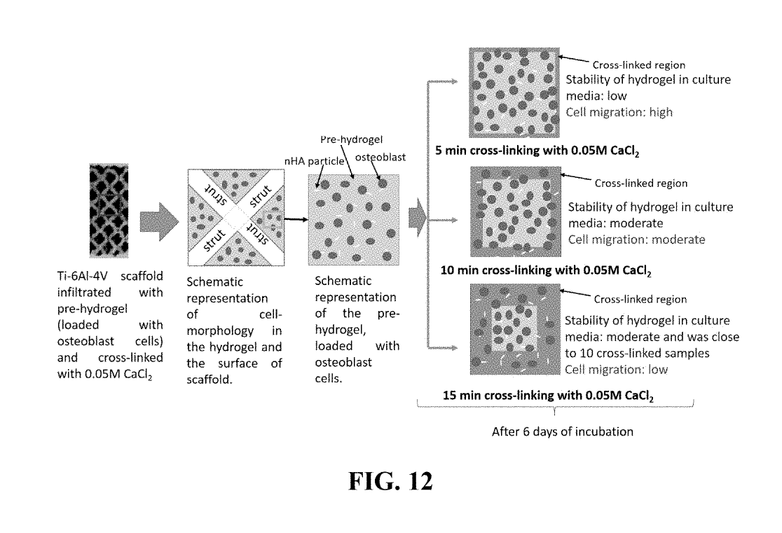

Effect of cross-linking time on cell viability. As mentioned in previous section, stability of hydrogel and hydroxyapatite-based nanocomposite depends on the cross-linking time. It was noted that 10 min cross-linking time was optimum with stable dissolution profile and no burst degradation of material. To further investigate the effect of cross-linking time on cell viability, cells were cultured for 6 days in a-MEM-based complete culture media at 37° C. and 5% CO₂ and 95% relative humidity. Old culture media was replaced with fresh media every second day. After 6 days, following live/dead assay, cells were stained to distinguish the live and dead cells (FIG. 11). In FIG. 11, broken lines show the struts of Ti-6Al-4V scaffold. Results show the presence live cells on the struts as well as in the pore region filled with cell loaded hydrogel in samples, cross-linked for 5 min (FIG. 11a), 10 min (FIGS. 11b), and 15 min (FIG. 11c). Only few dead cells were noticed on the struts as well as in the pore region. Interestingly, more uniform distribution of cells was noticed in the hydrogel matrix as well as on the struts in samples cross-linked for 5 and 10 min. This can be associated with presence of less stiff region, which allows easy migration of cells in the three-dimensional environment of hydrogel (FIG. 12). In contrast, in case of samples cross-linked for 15 min, a relatively lower number of cells were found in the vicinity of struts as compared to samples cross-linked for 10 min. This is due to slower migration of cells in 15 min cross-linked samples because of highly stiff hydrogel. Less number of cells were observed on the samples cross-linked for 5 min as compared to 10 min. Less number of cells in case of 5 min cross-linked samples can be a result of faster dissolution of samples as compared to 10 min cross-linked samples.

Due to the aforementioned reasons a higher number of cells were observed in the vicinity of struts in samples cross-linked for 10 min as compared to the samples cross-linked for 5 and 15 min.

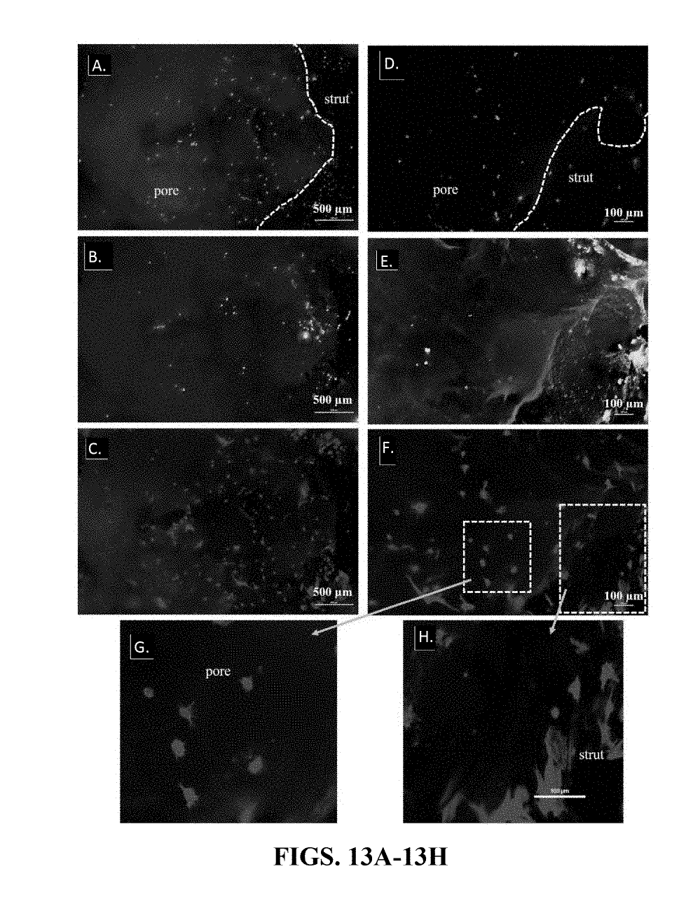

Cell viability and proliferation. The expression of prominent proteins, actin and vinculin as well as nucleus density was studied using immunofluorescence microscopy. In FIG. 13, broken lines show the struts of Ti-6Al-4V scaffold. Results indicate uniform staining of nucleus (FIG. 13a), vinculin (FIG. 13b), and actin filament (FIG. 13c). A higher cell density was noted in the vicinity of the strut as compared to hydrogel (FIG. 13d, 13e, 13f). This result is in good agreement with live/dead assay. Importantly, cells on strut were elongated and adopted the surface topography (FIG. 13h). However, in the pore region, filled with hydrogel, cells tend to adopt the three dimensional morphology (FIG. 13g).

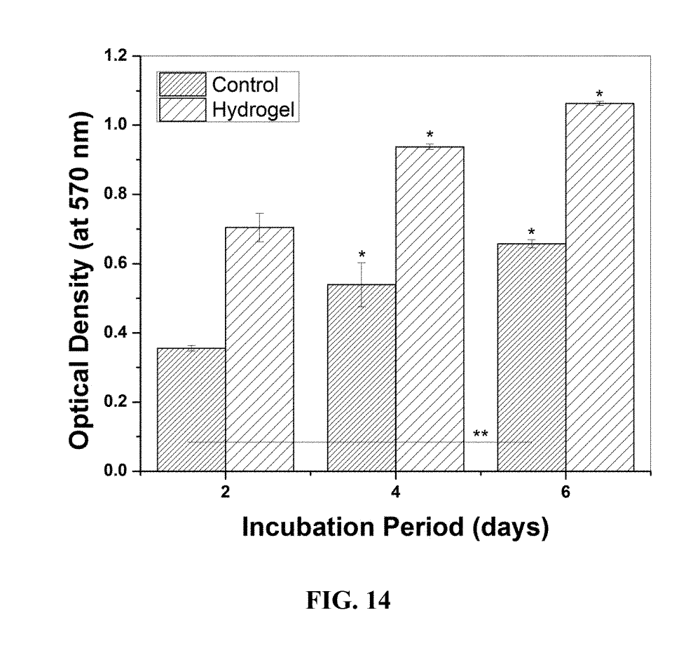

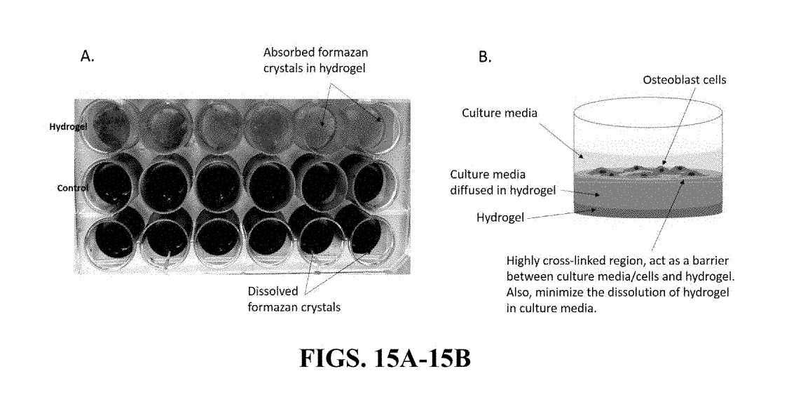

Furthermore, MTT assay was used to study cell proliferation because optical density is directly proportional to the metabolically active cells. The MTT assay was also used to compare the cell viability on samples. In the present study, in the individual group, an increase in cell viability with time was noted (FIG. 14). An increase in optical density with time is a clear indication of cell proliferation on both control and hydrogel. However, a decrease in the rate of proliferation was noted. Dunnett t test was used to compare the mean value of optical density on 4 and 6 days with mean value of optical density on 2 days at p<0.05. This is marked with symbol * on graph. In addition to this, Dunnett C test is used to compare the mean value of optical density among the samples mean at p<0.05. The Dunnett C comparison is marked with symbol **. Irrespective of the incubation time, a significant difference between hydrogel and control was observed, with higher cell density on hydrogel as compared to control at any point in time. Dunnett t test showed a significant difference between the samples when 4 and 6 days were compared with 2 days. Furthermore, a comparison among the samples using Dunnett t test showed a significant difference in optical density between the control samples incubated for 2 and 6 days. No such difference was noted in the case of hydrogel. It is of note that the optical density of hydrogel was normalized to avoid the false reading because a significant amount of formazan crystals were found absorbed in the hydrogel matrix. Related to this, FIG. 15a shows formazan crystals were absorbed in the hydrogel.

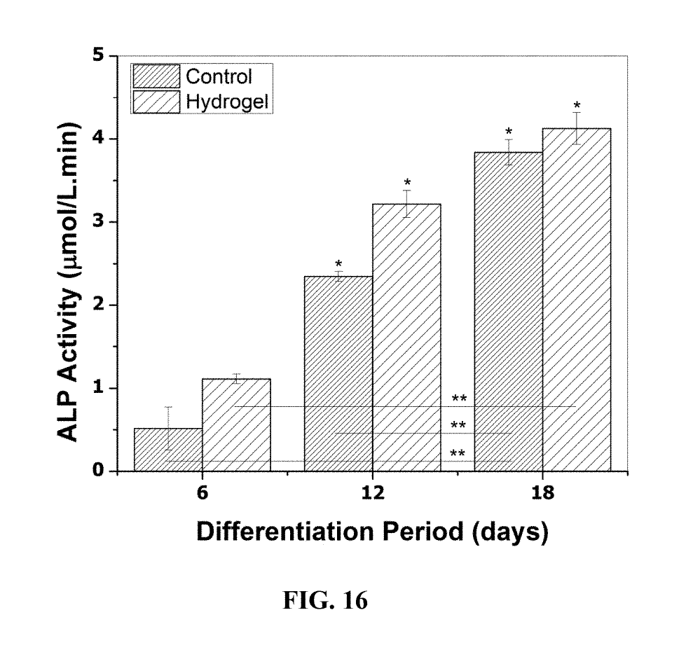

Alkaline phosphate activity. Alkaline phosphate activity (ALP) activity is a direct measure of activity of alkaline phosphatase enzyme. The higher value of ALP is associated with higher bone formation on implant. As mentioned above, optical density of the solution after 6, 12, and 18 days of differentiation was measured, followed by the calculation of ALP activity using equation 7. The calculation revealed a higher ALP activity on the hydrogel sample than control sample (FIG. 16). Dunnett t test (marked with symbol *) showed a significant difference in ALP activity between 6 and 12 days as well as 6 and 18 days on both control and hydrogel. Furthermore, Dunnett C test on control showed a significant difference between 6 and 18 day as well as 12 and 18 days. On hydrogel, Dunnett C test showed a significant difference between 6 and 18 days. After 6 days, as compared to control, ˜100% higher ALP activity was measured in hydrogel samples. Moreover, in the case of hydrogel, the value of ALP activity increased to ˜200% after 12 days as compared to 6 days. However, this value of ALP activity was higher than control on 12 days.

TABLE 1
Detail of composition used to prepare the hybrid hydrogel

| | Calculation for 20 ml hydrogel | | | | | |
|---|---|---|---|---|---|---|
| | | | Nano- | | | |
| | Alginate | Gelatin | hydroxyapatite | EDC | NHS | |
| Samples | (g) | (g) | (mg) | (mg) | (mg) | CaCl₂ |
| A | 0.2 | 0.2 | 100 | 50 | 30 | 0.05M |
| B | 0.2 | 0.2 | 20 | 50 | 30 | 0.05M |
| C | 0.2 | 0.1 | 20 | 50 | 30 | 0.05M |
| D | 0.1 | 0.2 | 20 | 50 | 30 | 0.05M |

1. An injectable hydrogel comprising:
(a) 0.005 to 0.02 g/ml alginate;
(b) 0.005 to 0.02 g/ml gelatin;
(c) 1 to 10 mg/ml nanocrystalline hydroxyapatite; and
(d) water to volume.

2. The hydrogel of claim 1, further comprising osteoblast or osteoblast precursor cells.

3. The hydrogel of claim 1, comprising 0.01 g/ml alginate.

4. The hydrogel of claim 1, comprising 0.01 g/ml gelatin.

5. The hydrogel of claim 1, comprising 5 mg/ml nanocrystalline hydroxyapatite.

6. The hydrogel of claim 1, wherein the hydrogel is crosslinked using CaCl₂.

7. The hydrogel of claim 1, further comprising 2 to 3 mg/ml EDC and 1 to 2 mg/ml NHS.

8. The hydrogel of claim 1, wherein the nanocrystalline hydroxyapatite is in the form of elongated particles having a length of about 80 nm and a diameter of about 30 nm.

9. A bone replacement implant comprising:
(a) a three dimensional support; and
(b) a hydrogel matrix of claim 1 comprising a hypoxia inducer and glucose;
wherein the implant is capable of promoting vascularization and osteogenesis.

10. The implant of claim 9, wherein the three dimensional support is a scaffold structure of Ti-6Al-4V.

11. The implant of claim 10, wherein the scaffold structure has a porosity of 50 to 70%.

12. The implant of claim 10, wherein the scaffold structure has an average pore size of 200 to 500 μm.

13. The implant of claim 10, wherein the scaffold structure has a thickness of 0.25 to 5 cm.

14. The implant of claim 10, wherein the scaffold structure has a density of 1 to 2 g/cm².

15. The implant of claim 9, wherein the hydrogel further comprises proteins of extracellular matrix.

16. The implant of claim 9, wherein the hydrogel further comprises natural, synthetic, or natural and synthetic polymers.

17. The implant of claim 16, wherein the natural polymers are one or more of polyhyaluronic acid, alginate, polypeptides, collagen, elastin, polylactic acid, polyglycolic acid, or chitin.

18. The implant of claim 16, wherein the synthetic polymers are one or more of polyethylene oxide, polyethylene glycol, polyvinyl alcohol, polyacrylic acid, polyacrylamide, poly(N-vinyl-2-pyrrolidone), polyurethane, or polyacrylonitrile.

19. The implant of claim 9, wherein the hydrogel further comprises one or more growth factors.

20. The implant of claim 9, wherein the hydrogel further comprises an antibiotic.

21. The implant of claim 9, wherein the hypoxia inducer is deferoxamine mesylate (DFM).

22. The implant of claim 21, wherein the DFM is present at a concentration of about 2 to 5 μM.

23. The implant of claim 9, further comprising a cell component.

24. The implant of claim 23, wherein the cell component comprises an osteoblast or osteoblast progenitor cell.
